(19) United States
(12) Patent Application Publication
(10) Pub. No.: US 2010/0099932 A1
(43) Pub. Date: Apr. 22, 2010

(54) Isoprene Compositions and Methods of Use

(75) Inventors: Gary A. Alianell, Villa Park, CA (US); Fredric Derwitsch, Melbourne, FL (US); Deborah Wells, Merritt Island, FL (US); Teresa Taylor, Melbourne, FL (US)

Correspondence Address: Mridula R. Pottathil, PhD, 1280 Highway A1A Unit F, Satellite Beach, FL, 32937, US

(73) Assignee: Ecoprene LLC, Melbourne, FL (US)

(21) Appl. No.: 12/603,378

(22) Filed: Oct. 21, 2009

Related U.S. Application Data
(60) Provisional application No. 61/107,009, filed on Oct. 21, 2008.

(51) Int. Cl. C07C 11/10

(52) U.S. Cl. 585/16

(57) ABSTRACT

The present invention provides fuel compositions comprising isoprene or renewable isoprene.

### FIELD OF THE INVENTION

The present invention relates to compositions comprising isoprene or renewable isoprene and methods of use of these compositions. The compositions and methods are useful in fuel and fuel blends and represents an alternative to current reliance on petroleum fuels.

### BACKGROUND

Isoprene is a common synonym for the chemical compound 2-methylbuta-1,3-diene. It is commonly used in industry and is an important biological material.

At room temperature, isoprene is a colorless liquid.

It is most readily available industrially as a by-product of the thermal cracking of naphtha or oil. About 95% of isoprene production is used to produce cis-1,4-polyisoprene—a synthetic version of natural rubber. This is used in the tire industry and represents a captive market, meaning potential customers in other areas are constrained to purchase isoprene because of limited supply.

Isoprene is formed naturally in animals and plants and is generally the most common hydrocarbon found in the human body. Isoprene is also common in low concentrations in many foods. Isoprene is produced in the chloroplasts of leaves of certain tree species through the DMAPP (Dimethylallyl pyrophosphate) pathway; the enzyme isoprene synthase is responsible for its biosynthesis. The amount of isoprene released from isoprene-emitting vegetation depends on leaf mass, leaf area, light (particularly photosynthetic photon flux density, or PPFD), and leaf temperature. Thus, during the night, little isoprene is emitted from tree leaves while daytime emissions are expected to be substantial (˜5-20 mg/m²/h) during hot and sunny days.

There are methods and compositions for the use of genetically modified microalgae, cyanobacteria, and photosynthetic and non-photosynthetic bacteria in the production and harvesting of 5-carbon volatile isoprenoid compounds, e.g., isoprene and methyl-butenol. Such genetically modified organisms can be used commercially in an enclosed mass culture system, e.g., to provide a source of renewable fuel for internal combustion engines or, upon on-board reformation, in fuel-cell operated engines; or to provide a source of isoprene for uses in other chemical processes such as chemical synthesis.

### Traditional Petroleum Fuels

Gasoline (gas) or petroleum spirit (petrol) is a petroleum-derived liquid mixture consisting mostly of aliphatic hydrocarbons, enhanced with iso-octane or the aromatic hydrocarbons toluene and benzene to increase its octane rating, and is primarily used as fuel in internal combustion engines.

Gasoline is a mixture of hydrocarbons, although some may contain significant quantities of ethanol and some may contain small quantities of additives such as methyl tert-butyl ether as anti-knock agents to increase the octane rating. The hydrocarbons consist of a mixture of n-paraffins, naphthenes, olefins and aromatics. Naphthenes, olefins and aromatics increase the octane rating of the gasoline whereas the n-paraffins have the opposite effect

Gasoline is produced in oil refineries. Material that is separated from crude oil via distillation, called virgin or straight-run gasoline, does not meet the required specifications for modern engines (in particular octane rating), but will form part of the blend.

The bulk of a typical gasoline consists of hydrocarbons with between 5 and 12 carbon atoms per molecule.

The various refinery streams blended together to make gasoline all have different characteristics. Some important streams are:
Reformate, produced in a catalytic reformer with a high octane rating and high aromatic content, and very low olefins (alkenes).
Cat Cracked Gasoline or Cat Cracked Naphtha, produced from a catalytic cracker, with a moderate octane rating, high olefins (alkene) content, and moderate aromatics level. Here, “cat” is short for “catalytic”.
Hydrocrackate (Heavy, Mid, and Light), produced from a hydrocracker, with medium to low octane rating and moderate aromatic levels.
Virgin or Straight-run Naphtha (has many names), directly from crude oil with low octane rating, low aromatics (depending on the crude oil), some naphthenes (cycloalkanes) and no olefins (alkenes).
Alkylate, produced in an alkylation unit, with a high octane rating and which is pure paraffin (alkane), mainly branched chains.
Isomerate (various names) which is obtained by isomerising the pentane and hexane in light virgin naphthas to yield their higher octane isomers.
   Overall a typical gasoline is predominantly a mixture of paraffins (alkanes), naphthenes (cycloalkanes), and olefins (alkenes). The exact ratios can depend on
the oil refinery that makes the gasoline, as not all refineries have the same set of processing units.
the crude oil feed used by the refinery.
the grade of gasoline, in particular the octane rating.

Currently many countries set tight limits on gasoline aromatics in general, benzene in particular, and olefin (alkene) content. This is increasing the demand for high octane pure paraffin (alkane) components, such as alkylate, and is forcing refineries to add processing units to reduce the benzene content.

Gasoline can also contain some other organic compounds: such as organic ethers (deliberately added), plus small levels of contaminants, in particular sulfur compounds such as disulfides and thiophenes. Some contaminants, in particular thiols and hydrogen sulfide, must be removed because they cause corrosion in engines. Sulfur compounds are usually removed by hydrotreating, yielding hydrogen sulfide which can then be transformed into elemental sulfur via the Claus process.

Gasoline is more volatile than diesel oil; Jet-A or kerosene, not only because of the base constituents, but because of the additives that are put into it. The final control of volatility is often achieved by blending with butane. The Reid Vapor Pressure test is used to measure the volatility of gasoline. The desired volatility depends on the ambient temperature: in hotter climates, gasoline components of higher molecular weight and thus lower volatility are used. In cold climates, too little volatility results in cars failing to start. In hot climates, excessive volatility results in what is known as “vapor lock” where combustion fails to occur, because the liquid fuel has changed to a gaseous fuel in the fuel lines, rendering the fuel pump ineffective and starving the engine of fuel.

An important characteristic of gasoline is its octane rating, which is a measure of how resistant gasoline is to the abnormal combustion phenomenon known as detonation (also known as knocking, pinging, spark knock, and other names). Deflagration is the normal type of combustion. Octane rating is measured relative to a mixture of 2,2,4-trimethylpentane (an isomer of octane) and n-heptane. There are a number of different conventions for expressing the octane rating; therefore, the same fuel may be labeled with a different number, depending upon the system used.

### Lead

The mixture known as gasoline, when used in high compression internal combustion engines, has a tendency to autoignite (detonation) causing a damaging “engine knocking” (also called “pinging”) noise. The discovery that lead additives modified this behavior led to the widespread adoption of the practice in the 1920s and therefore more powerful higher compression engines. The most popular additive was tetra-ethyl lead. However, with the discovery of the environmental and health damage caused by the lead, and the incompatibility of lead with catalytic converters found on virtually all newly sold US automobiles since 1975, this practice began to wane (encouraged by many governments introducing differential tax rates) in the 1980s. Most countries are phasing out leaded fuel; different additives have replaced the lead compounds. The most popular additives include aromatic hydrocarbons, ethers and alcohol (usually ethanol or methanol).

Gasoline, as delivered at the pump, also contains additives to reduce internal engine carbon buildups, improve combustion, and to allow easier starting in cold climates.

### MMT

Methylcyclopentadienyl manganese tricarbonyl (MMT) has been used for many years in Canada and recently in Australia to boost octane. It also helps old cars designed for leaded fuel run on unleaded fuel without need for additives to prevent valve problems.

US Federal sources state that MMT is suspected to be a powerful neurotoxin and respiratory toxin, and a large Canadian study concluded that MMT impairs the effectiveness of automobile emission controls and increases pollution from motor vehicles.

In 1977 use of MMT was banned in the US by the Clean Air Act until the Ethyl Corporation could prove that the additive would not lead to failure of new car emissions-control systems. As a result of this ruling, the Ethyl Corporation began a legal battle with the EPA, presenting evidence that MMT was harmless to automobile emissions-control systems. In 1995 the US Court of Appeals ruled that the EPA had exceeded its authority, and MMT became a legal fuel additive in the US. MMT is nowadays manufactured by the Afton Chemical Corporation division of Newmarket Corporation.

### Oxygenate Blending

Oxygenate blending adds oxygen to the fuel in oxygen-bearing compounds such as MTBE, ETBE and ethanol, and so reduces the amount of carbon monoxide and unburned fuel in the exhaust gas, thus reducing smog. In many areas throughout the US oxygenate blending is mandated by EPA regulations to reduce smog and other airborne pollutants. For example, in Southern California, fuel must contain 2% oxygen by weight, resulting in a mixture of 5.6% ethanol in gasoline. The resulting fuel is often known as reformulated gasoline (RFG) or oxygenated gasoline.

### Ethanol Fuel Mixtures

Ethanol and to a lesser extent the ethanol derived ETBE are a common replacements for MTBE. Especially since ethanol derived from biomatter such as corn, sugar cane or grain is frequent, this will often be referred to as bio-ethanol. A common ethanol-gasoline mix of 10% ethanol mixed with gasoline is called gasohol or E10, and an ethanol-gasoline mix of 85% ethanol mixed with gasoline is called E85.

To avoid engine stall due to “slugs” of water in the fuel lines interrupting fuel flow, the fuel must exist as a single phase. The fraction of water that an ethanol-gasoline fuel can contain without phase separation increases with the percentage of ethanol. This shows, for example, that E30 can have up to about 2% water. If there is more than about 71% ethanol, the remainder can be any proportion of water or gasoline and phase separation will not occur. However, the fuel mileage declines with increased water content. The increased solubility of water with higher ethanol content permits E30 and hydrated ethanol to be put in the same tank since any combination of them always results in a single phase.

### Renewable Energy

Renewable energy is energy generated from natural resources—such as sunlight, wind, rain, tides and geothermal heat—which are renewable (naturally replenished). Renewable energy technologies range from solar power, wind power, hydroelectricity/micro hydro, biomass and biofuels for transportation.

### Liquid Biofuel

Liquid biofuel is usually either a bioalcohol such as ethanol fuel or a bio-oil such as biodiesel and straight vegetable oil. Biodiesel can be used in modern diesel vehicles with little or no modification to the engine and can be made from waste and virgin vegetable and animal oil and fats (lipids). Virgin vegetable oils can be used in modified diesel engines. In fact the Diesel engine was originally designed to run on vegetable oil rather than fossil fuel. A major benefit of biodiesel is lower emissions. The use of biodiesel reduces emission of carbon monoxide and other hydrocarbons by 20 to 40%.

In some areas corn, cornstalks, sugarbeets, sugar cane, and switchgrasses are grown specifically to produce ethanol (also known as grain alcohol) a liquid which can be used in internal combustion engines and fuel cells. Ethanol is being phased into the current energy infrastructure. E85 is a fuel composed of 85% ethanol and 15% gasoline that is sold to consumers. Biobutanol is being developed as an alternative to bioethanol.

There is growing international criticism about biofuels from food crops with respect to issues such as food security, environmental impacts (deforestation) and energy balance.

Ethanol fuel is ethanol (ethyl alcohol), the same type of alcohol found in alcoholic beverages. It can be used as a fuel, mainly as a biofuel alternative to gasoline, and is widely used in cars in Brazil. Because it is easy to manufacture and process, and can be made from very common crops, such as sugar cane and maize (corn), it is an increasingly common alternative to gasoline in some parts of the world.

Anhydrous ethanol (ethanol with less than 1% water) can be blended with gasoline in varying quantities up to pure ethanol (E100), and most spark-ignited gasoline style engines will operate well with mixtures of 10% ethanol (E10). Most cars on the road today in the U.S. can run on blends of up to 10% ethanol, and the use of 10% ethanol gasoline is mandated in some cities where harmful levels of auto emissions are possible.

Ethanol can be mass-produced by fermentation of sugar or by hydration of ethylene (ethene CH₂═CH₂) from petroleum and other sources. Current interest in ethanol mainly lies in bio-ethanol, produced from the starch or sugar in a wide variety of crops, but there has been considerable debate about how useful bio-ethanol will be in replacing fossil fuels in vehicles. Concerns relate to the large amount of arable land required for crops, as well as the energy and pollution balance of the whole cycle of ethanol production.

Ethanol is considered “renewable” because it is primarily the result of conversion of the sun's energy into usable energy. Creation of ethanol starts with photosynthesis causing the feedstocks such as switchgrass, sugar cane, or corn to grow. These feedstocks are processed into ethanol.

Current, first generation processes for the production of ethanol from corn use only a small part of the corn plant: the corn kernels are taken from the corn plant and only the starch, which represents about 50% of the dry kernel mass, is transformed into ethanol. Two types of second generation processes are under development. The first type uses enzymes and yeast to convert the plant cellulose into ethanol while the second type uses pyrolysis to convert the whole plant to either a liquid bio-oil or a syngas. Second generation processes can also be used with plants such as grasses, wood or agricultural waste material such as straw.

There is an enormous need for alternatives to petroleum based fuels that do not rely on food crops. However, there is also the fact that a completely re-designed engine to handle a purely bio-based or non-petroleum based fuel is unrealistic. It is therefore the goal of the present invention to disclose fuel blends that comprise isoprene or isoprene made from a renewable source that can be combined with petroleum in varying amounts. This will help reduce our use of petroleum fuels.

### SUMMARY OF THE INVENTION

### Renewable Isoprene

Current production of isoprene is as a by-product of the thermal cracking of naptha or oil. Given current issues with limited oil supplies, there is a need for a renewable source of isoprene.

The amount of isoprene produced by plants is far too low to collect cost efficiently. The isoprene synthesis pathway from the poplar plant has been genetically engineered to express isoprene in a variety of microorganisms.

There are methods and compositions for the use of genetically modified microalgae, cyanobacteria, and photosynthetic and non-photosynthetic bacteria in the production and harvesting of 5-carbon volatile isoprenoid compounds, e.g., isoprene and methyl-butenol. Such genetically modified organisms can be used commercially in an enclosed mass culture system, e.g., to provide a source of renewable fuel for internal combustion engines or, upon on-board reformation, in fuel-cell operated engines; or to provide a source of isoprene for uses in other chemical processes such as chemical synthesis.

The present invention relates to use of isoprene—currently in limited supply—in a fuel blend. This represents but is not limited to a replacement for ethanol in fuel blends. In addition, the present invention relates blends of isoprene in ethanol that can be used in petroleum fuel blends to enhance the performance of ethanol in those blends.

The current issue in use of ethanol in fuel blends has been use of food crops as a feedstock from which ethanol is derived. This has increased food prices and has had an overall detrimental effect on the world food chain. Therefore, it would be advantageous to find a truly renewable replacement for ethanol in fuel blends.

The present invention discloses compositions comprising renewable isoprene. The renewable isoprene can be combined with other hydrocarbons, petroleum fuels, or with ethanol to produce a biofuel.

### BRIEF DESCRIPTION OF THE DRAWINGS

FIG. 1 depicts a chart showing a few of the chemical and physical properties of isoprene.

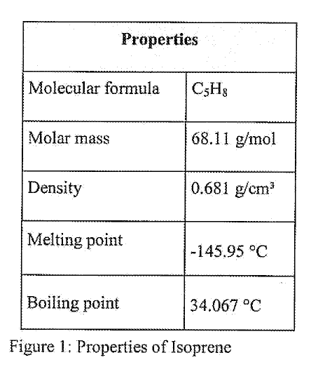

### DETAIL DESCRIPTION OF THE INVENTION

The present invention comprises renewable isoprene. One embodiment of the invention is a composition comprising isoprene in petroleum to produce a fuel blend. Examples of renewable isoprene include but are not limited to isoprene produced by microorganisms, yeast, or fungi expressing genes that allow for expression of isoprene production components. There are methods and compositions for the use of genetically modified microalgae, cyanobacteria, and photosynthetic and non-photosynthetic bacteria in the production and harvesting of 5-carbon volatile isoprenoid compounds, e.g., isoprene and methyl-butenol. Such genetically modified organisms can be used commercially in an enclosed mass culture system, e.g., to provide a source of renewable fuel for internal combustion engines or, upon on-board reformation, in fuel-cell operated engines; or to provide a source of isoprene for uses in other chemical processes such as chemical synthesis.

Examples of a petroleum fuel include but are not limited to gasoline, diesel, pentane, and butane.

Another embodiment of the invention is a method of polymer production using renewable isoprene.

Another embodiment of the invention is an ethanol fuel composition comprising isoprene.

Another embodiment of the invention is a gasoline composition comprising isoprene or renewable isoprene as a replacement for butane stabilizer for volatility control.

Another embodiment of the invention is the use of isoprene as a direct replacement for gasoline.

Another embodiment of the invention is the use of isoprene as a direct replacement for propane, butane, Liquefied petroleum gas (LPG), or Liquefied natural gas (LNG) (gas phase)

Another embodiment of the invention is the use of renewable isoprene as a solvent.

Another embodiment of the invention is the use of renewable isoprene as a retardant.

Another embodiment of the invention is the use of renewable isoprene as a lubricant.

1. A fuel composition comprising isoprene.

2. The composition of claim 1 wherein the isoprene is produced from a genetically modified microorganism.

3. A fuel composition comprising isoprene and ethanol.

4. A fuel composition comprising isoprene and a petroleum fuel wherein the petroleum fuel is gasoline, diesel, methane, ethane, butane, pentane, hexane, or heptane.
